(19) United States
(12) Patent Application Publication
(10) Pub. No.: US 2004/0210069 A1
(43) Pub. Date: Oct. 21, 2004

(54) Process for preparing organic hydroperoxides

(76) Inventors: Wan Shi Foong, Singapore (SG); Raymond Lawrence June, Singapore (SG)

Correspondence Address: Jennifer D. Adamson, Shell Oil Company, Legal - Intellectual Property, P. O. Box 2463, Houston, TX, 77252-2463, US

(21) Appl. No.: 10/789,173

(22) Filed: Feb. 27, 2004

(30) Foreign Application Priority Data
Feb. 28, 2003 (SG) 200300833-1

(51) Int. Cl. C07D 301/19

(52) U.S. Cl. 549/529

(57) ABSTRACT

The invention relates to a process for preparing organic hydroperoxides, which process involves (a) oxidizing an organic compound to obtain a reaction product containing organic hydroperoxide, (b) treating at least part of the organic hydroperoxide containing reaction product with a basic aqueous solution and separating hydrocarbonaceous phase containing organic hydroperoxide from basic aqueous phase, (c) washing with water at least part of the separated hydrocarbonaceous phase containing organic hydroperoxide and separating hydrocarbonaceous phase containing organic hydroperoxide from aqueous phase, and (d) optionally repeating step (c) one or more times, in which process solid particles are removed from the reaction product containing organic hydroperoxide and/or basic aqueous solution before use in step (b), preferably by filtering. The invention also relates to a process for preparing oxirane compounds in which such organic hydroperoxide is used.

### FIELD OF THE INVENTION

The present invention relates to a process for preparing organic hydroperoxides and to processes in which such organic hydroperoxides are used, such as the preparation of oxirane compounds and preparation of alkenyl aryl compound.

### BACKGROUND OF THE INVENTION

Processes for preparing propylene oxide employing organic hydroperoxides are known in the art. As described in U.S. Pat. No. 5,883,268, such process conventionally comprises peroxidation of ethylbenzene, followed by contacting the peroxidation reaction product with aqueous base in an amount sufficient to neutralize acidic components thereof and separating the resulting mixture into an aqueous stream and a deacidified organic stream. The base contaminated, deacidified hydroperoxide stream is washed with water and the resulting mixture separated into an organics contaminated water phase and an organic phase having a reduced alkali metal content.

It would be useful to find a process that would reduce the amount of contaminants in the final organic hydroperoxide obtained in a simple and effective way.

### SUMMARY OF THE INVENTION

The present invention is directed to a process for preparing organic hydroperoxides, which process comprises:

(a) oxidizing an organic compound to obtain a reaction product containing organic hydroperoxide,

(b) treating at least part of the reaction product with a basic aqueous solution and separating hydrocarbonaceous phase containing organic hydroperoxide from basic aqueous phase;

(c) washing with water at least part of the separated hydrocarbonaceous phase containing organic hydroperoxide and separating hydrocarbonaceous phase containing organic hydroperoxide from aqueous phase; and,

(d) optionally repeating step (c) one or more times, which process further comprises removing solid particles from the reaction product containing organic hydroperoxide and/or basic aqueous solution before use in step (b).

Organic hydroperoxides are useful in a range of processes. One of these processes is the reaction of organic hydroperoxide with olefin in order to obtain oxirane compounds. In such process, the organic compound usually is an alkylaryl compound, and the process further comprises:

(e) contacting at least part of the hydrocarbonaceous phase containing alkylaryl hydroperoxide obtained in step (c) and/or (d) with olefin and catalyst to obtain alkylaryl hydroxide and oxirane compounds, and

(f) separating at least part of the oxirane compound from the alkylaryl hydroxide.

The alkylaryl hydroxide obtained in step (f) may /e used in a wide range of processes. Such process is preparing an alkenyl aryl compound by dehydrating the alkylaryl hydroxide. Another process is hydrogenating the alkylaryl hydroxide to obtain an alkylaryl compound. If the process according to the present invention is to be used for dehydrating the alkylaryl hydroxide, the process suitably comprises further:

(g) converting at least part of the alkylaryl hydroxide obtained in step (f).

### DETAILED DESCRIPTION OF THE INVENTION

Although the organic compound used in the process of the present invention may in principle be any compound, organic compounds which are most frequently used are alkylaryl compounds, more specifically benzene compounds containing at least 1 alkyl substituent which alkyl substituent contains of from 1 to 10 carbon atoms, preferably of from 2 to 8 carbon atoms. Preferably, the benzene compound contains on average of from 1 to 2 constituents. The alkylaryl compounds most frequently encountered are ethylbenzene, cumene and di(iso-propyl)benzene.

The oxidation of the organic compound may be carried out by any suitable process known in the art. The oxidation may be carried out in the liquid phase in the presence of a diluent. This diluent is preferably a compound which is liquid under the reaction conditions and does not react with the starting materials and product obtained. However, the diluent may also be a compound necessarily present during the reaction. For example, if the alkylaryl compound is ethylbenzene the diluent may be ethylbenzene as well.

Besides the desired organic hydroperoxide, a wide range of contaminants are created during the oxidation of organic compounds. Although most of these are present in small amounts, the presence of organic acids has been found to sometimes cause problems in the further use of the organic hydroperoxides. As described in U.S. Pat. No. 5,883,268, a method of reducing the amount of contaminants is contacting the reaction product containing organic hydroperoxide with an aqueous alkali solution. However, contact with the aqueous alkali solution introduces a certain amount of alkali metal into the organic hydroperoxide containing reaction product. The amount of organic acids is decreased by an alkali wash but the amount of alkali metal contaminants is increased.

In the process of the present invention, the organic hydroperoxide containing reaction product is contacted with a basic aqueous solution, more specifically a basic aqueous solution comprising one or more alkali metal compounds. Suitable alkali sources for use in the aqueous alkali solution include alkali metal hydroxides, alkali metal carbonates and alkali metal hydrogen carbonates. Examples of these compounds are NaOH, KOH, Na₂CO₃, K₂CO₃, NaHCO₃ and KHCO₃. In view of their easy availability, it is preferred to use NaOH and/or Na₂CO₃.

The basic aqueous solution preferably contains fresh basic aqueous solution, recycled basic aqueous solution and optionally additional water. The recycled basic aqueous solution may be obtained from step (b).

The conditions under which step (b) is carried out, depend on the further circumstances. Preferably, step (b) is carried out at a temperature of between 0° C. and 150° C., more preferably of between 20° C. and 100° C.

In step (b), the hydrocarbonaceous phase is subsequently separated from the aqueous phase. A preferred method comprises allowing the hydroarbonaceous phase and aqueous phase to settle in a settling vessel and subsequently separating a hydrocarbonaceous phase from an aqueous phase. Preferably, the hydrocarbonaceous phase containing organic hydroperoxide is subsequently sent to a coalescer where further aqueous phase is removed. Preferably, the separation is carried out at a temperature between 0° C. and 150° C., more preferably between 20° C. and 100° C.

In conventional operations, an interface emulsion layer or rag may sometimes be observed upon washing the hydrocarbonaceous phase containing organic hydroperoxide. Surprisingly, it has now been found that such rag formation may be prevented by removing solid particles from one or more of the feed streams used in step (b). Without wishing to be bound to any theory, it is thought that rag formation is due to the presence of solid particles, such as small, insoluble particles of metal compounds such as iron. Such metal compounds may be formed in the corrosion of metal surfaces. The organic hydroeroxide containing reaction product could pick up such metal compounds during oxidation. Furthermore, the stream containing the organic compound may already contain such solid particles before the oxidation. The basic aqueous solution may pick up such metal compounds from recycled basic aqueous solution and/or from waste water which is used in the preparation of the basic aqueous solution. Both feed streams may pick up metal compounds during storage. Solid particles may be removed in different ways. Solid particles may be removed in any way known to someone skilled in the art. Suitable methods comprise treating at least part of one or more of the feed streams used in step (b) with an ion exchange resin, with an adsorbent and/or filtering at least part of these feed streams. Filtering is the preferred method of removing solid particles. Preferably, the solid particles are removed by filtering at least part of the reaction product containing organic hydroperoxide and/or at least part of the basic aqueous solution before use in step (b). Such layer may cause an increased amount of basic aqueous solution in the organic phase. The presence of a substantial amount of basic aqueous solution in the organic phase tends to cause problems in the further processing of the organic hydroperoxide mainly caused by the presence of basic compounds, more specifically compounds such as sodium hydroxide and calcium hydroxide.

The temperature and pressure at which the filtering may be carried out are known to someone skilled in the art and depend on the compounds present.

The filter which is preferably used for filtering the feed streams of step (b) has openings of 50 micrometres or less, preferably 30 micrometres or less, more preferably 20 micrometres or less.

The filter may be made of any material which is known to be suitable by someone skilled in the art. Preferred materials are polypropylene and cellulose. Filters may slowly plug during use which is shown by an increased pressure drop over the filter. When the pressure drop becomes too high, the filter may be taken out of operation, cleaned and be returned as known to someone skilled in the art. Alternatively, the filter may be cleaned by feeding a clean liquid such as cumene or ethylbenzene in the reverse direction of the normal flow, so-called back-flushing. The latter has the advantage that the filter does not need to be removed.

As mentioned above, each of the feed streams which is used in step (b) may contain metal compounds which need to be removed. The amount of metal compounds which is incorporated in the organic hydroperoxide containing reaction product produced in step (a) depends on the amount of metal compounds present in the organic compound subjected to step (a) and on the exact processing conditions in step (a). The amount and kind of metal compounds present in step (a) will determine whether solid particles need to be removed from the product of step (a), or part of it.

The basic aqueous solution used in step (b) may pick up metal compounds from various sources. The amount of metal compounds present in each source will determine when solid particles are preferably removed from the basic aqueous phase.

In order to further improve the separation of aqueous phase and hydrocarbonaceous phase in steps (b), (c) and/or optional step (d), additional compounds may be present. Examples of such additional compounds are so-called emulsion breakers or de-hazers such as aliphatic or cyclic amines.

In the present description of the invention, the expression water is used to indicate both clean water and waste water which may contain contaminants. If clean water is to be used, this is mentioned separately. The washing with water of steps (c) and (d) may be carried out with clean water and/or waste water.

Waste water used for washing, optionally in combination with clean water, has many advantages. This has been described extensively in not-prepublished patent application PCT/EP02/10519. Therefore, the washing with water of steps (c) and/or (d) preferably is carried out with waste water optionally in combination with clean water.

The waste water may be added to separated hydrocarbonaceous phase at any stage. A preferred, specific embodiment comprises adding waste water or aqueous solution containing waste water to a coalescer.

Preferably, the water used in step (c) and/or (d) comprises both waste water previously used in washing a hydrocarbonaceous phase containing organic hydroperoxide and a different kind of waste water.

The waste water previously used in washing a hydrocarbonaceous phase containing organic hydroperoxide, preferably is a waste water obtained by contacting a hydrocarbonaceous phase containing organic hydroperoxide with an aqueous phase, preferably clean water, and subsequently separating the aqueous phase from the hydrocarbonaceous phase. The aqueous phase so obtained is preferably used as waste water without further treatment. Most preferably, the waste water obtained in this way is used in combination with a different kind of waste water.

The washing of the hydrocarbonaceous phase is preferably carried out by contacting the hydrocarbonaceous phase countercurrently with water. Countercurrent operation is considered to comprise contacting with relatively clean water hydrocarbonaceous phase which has already been washed once or more, while contacting hydrocarbonaceous phase which has not yet been washed, with aqueous phase which already has been in contact with hydrocarbonaceous phase.

The source of waste water is in principle irrelevant to the present process. However, it is preferred that the waste water is obtained in a process step related to the present process as this reduces the risk that the compounds present in the hydrocarbonaceous phase react with those present in the aqueous solution. Furthermore, it is preferred not to introduce new components into the process. It is surprising that the use of waste water gives good results as the aim of the previous process steps was to remove organic acids which were formed as by-products in the oxidation of step (a). It has now been found that waste water may be used in the aqueous wash of step (c) and/or (d), giving good results without negative impact on a subsequent catalyst such as an epoxidation catalyst such as described in EP-A-345856.

Waste water which has been found especially suitable for use in aqueous solutions for the present invention is waste water which is acidic. Preferably, the acidic waste water comprises one or more organic acids. Organic acids have been found to be generally compatible with the compounds further used in the present process. It has been found especially preferred if the acid which is present is an organic acid comprising from 1 to 20 carbon atoms. Preferred organic acids to be present in the waste water include hydrocarbyl carboxylic acids having in total from 1 to 10 carbon atoms. Especially preferred acids are formic acid, acetic acid, propionic acid and butyric acid. It has been found that formic acid is especially suitable as formic acid was observed to give only limited decomposition of the organic hydroperoxide.

The concentration of acid in the aqueous solution preferably is from 0.0001% wt to 5% wt, based on total amount of aqueous solution, more preferably from 0.001% wt to 2% wt, most preferably from 0.001% wt to 1% wt.

Preferably, the water for use in steps (c) and/or (d) consists of waste water optionally in combination with clean water and has a pH of from 2 to 7, preferably of from 3 to less than 7, more preferably of from 3.5 to 6.5.

Waste water streams may be used without further processing. However, in some cases it might be advantageous to concentrate the waste water stream before use in the process according to the present invention.

Dependent on the amount of contaminants present in the hydrocarbonaceous phase containing organic peroxide, water wash is either carried out once or a number of times. Preferably, the washing is carried out of from 1 to 3 times.

In optional process step (e), at least part of the hydrocarbonaceous phase containing organic hydroperoxide obtained in steps (c) and/or (d) is contacted with olefin, preferably propene, in the presence of a catalyst to obtain alkylaryl hydroxide and oxirane compounds. A catalyst which may suitably used in such process comprises titanium on silica and/or silicate. A preferred catalyst is described in EP 345856. The reaction generally proceeds at moderate temperatures and pressures, in particular at temperatures in the range of from 0° C. to 200° C., preferably in the range from 25° C. to 200° C. The precise pressure is not critical as long as it suffices to maintain the reaction mixture as a liquid or as a mixture of vapor and liquid. Atmospheric pressure may be satisfactory. In general, pressures may be in the range of from 1 to 100×10⁵ N/m².

The oxirane compounds may be separated from the reaction product containing alkylaryl hydroxide in any way known to be suitable to someone skilled in the art. The liquid reaction product may be worked up by fractional distillation, selective extraction and/or filtration. The solvent, the catalyst and any unreacted olefin or alkylaryl hydroperoxide may be recycled for further utilization.

Preferably, step (g) comprises either dehydration or hydrogenolysis of the reaction product. Hydrogenolysis is the reaction of the alkylaryl hydroxide with hydrogen, preferably in the presence of catalyst. Dehydration will generally produce an alkenyl aryl compound and water, while hydrogenolysis will generally produce alkylaryl compound. Preferably, the hydrogenolysis will produce the alkylaryl compound used as starting compound.

The alkylaryl hydroxide obtained in the process may be dehydrated in the presence of a catalyst to obtain styrene and water. Processes which may be used for this step have been described in WO 99/42425 and WO 99/42426. However, any suitable process known to someone skilled in the art may in principle be used.

The present invention is further illustrated by the following non-limiting examples.

### COMPARATIVE EXAMPLE 1

In a reactor, air was blown through ethylbenzene. The product obtained contained ethylbenzene hydroperoxide. This product was contacted with a solution containing 0.5% wt NaOH in water and mixed at a temperature of 60° C. The weight ratio of product containing ethylbenzene hydroperoxide to NaOH containing solution was 4.5:1 (wt:wt). The neutralized mixture obtained was sent to a settling vessel where a neutralized hydrocarbonaceous phase containing ethylbenzene hydroperoxide was separated from an aqueous phase.

The neutralized hydrocarbonaceous phase containing ethylbenzene hydroperoxide was sent to a coalescer where further aqueous phase was removed. The neutralized hydrocarbonaceous phase containing ethylbenzene hydroperoxide was washed by mixing the neutralized ethylbenzene hydroperoxide solution from the coalescer with an aqueous solution, separating the mixture obtained in a settling vessel into an aqueous phase and a hydrocarbonaceous phase, subsequently separating the hydrocarbonaceous phase obtained from the settling vessel with the help of a first coalescer, and separating the hydrocarbonaceous phase obtained in the first coalescer with the help of a second coalescer. Each of these steps is described in more detail herein below. The hydrocarbonaceous phase obtained in the second coalescer contained ethylbenzene hydroperoxide, ethyl benzene, water and contaminants. This hydrocarbonaceous phase was distilled. The distillate contained ethyl benzene, water and contaminants. This distillate was phase separated in a vessel to obtain a hydrocarbonaceous phase containing ethyl benzene and contaminants, and an aqueous phase containing water and contaminants. The latter had a pH of 3 and was used as wastewater for use in the aqueous solution for washing the neutralized hydrocarbonaceous phase.

The neutralized ethylbenzene hydroperoxide solution was mixed with an aqueous solution in a ratio of 4.5:1 (wt:wt). The aqueous solution comprised 85% wt of water which was recycled in this process step to which is added 1.3% wt of clean water and 13.7% wt of wastewater which had been used in washing a hydrocarbonaceous phase containing organic hydroperoxide.

The mixture which was obtained was sent to a settling vessel where a hydrocarbonaceous phase was separated from an aqueous phase.

NaOH was added to the aqueous phase obtained, which NaOH containing aqueous phase was for use in the neutralization of the hydrocarbonaceous phase containing ethylbenzene hydroperoxide.

The hydrocarbonaceous phase obtained in the settler, was sent to a first coalescer where were added 1.1% wt (based on total hydrocarbonaceous phase) of the distillate aqueous phase containing water and contaminants described above, and 1.7% wt (based on total hydrocarbonaceous phase) of clean water. An aqueous phase and a hydrocarbonaceous phase were obtained in the first coalescer. The hydrocarbonaceous phase from the first coalescer was sent to the second coalescer where further 1.4% wt (based on total hydrocarbonaceous phase) of clean water were added.

It was found that the hydrocarbonaceous phase obtained from the second coalescer, contained about 1 ppm of sodium.

### EXAMPLE 1

Comparative Example 1 was repeated except that the NaOH containing aqueous phase which was recycled to the neutralization step was filtered with a Whatman polypropylene filter having openings of at most 0.4 micrometers before being used again in the neutralization step.

It was found that the hydrocarbonaceous phase obtained from the second coalescer contained substantially less than 1 ppm of sodium. Additionally, it was found that the pressure over the filter gradually increased from 0.05×10⁵ N/m² to 1×10⁵ N/m² in the course of 3 weeks. This indicates that solids were being separated off.

### EXAMPLE 2

Waste water was obtained in the dehydration of 1-phenyl ethanol to styrene. The waste water obtained was distilled whereby the distillate obtained contained water and organic compounds. Organic phase was separated off from the distillate in a settler. The aqueous phase was sent from the settler to a coalescer. The aqueous phase obtained in the coalescer contained 10 ppm of solids of which 2 ppm was iron. To this aqueous phase was added 20% wt of NaOH. The NaOH solution thus obtained was filtered with a polypropylene filter having openings of different maximum sizes. The filtrate was contacted with a solution of ethylbenzene hydroperoxide in ethylbenzene at 70° C. for several hours. In a comparative example, the NaOH solution had not been filtered before use. The following results were obtained.

| filter size | | | | | |
|---|---|---|---|---|---|
| (micrometre) | none | 40 | 20 | 10 | 6 |
| rag formation | strong | slight | none | none | none |

We claim:

1. A process for preparing organic hydroperoxides, which process comprises:
(a) oxidizing an organic compound to obtain a reaction product containing organic hydroperoxide;
(b) treating at least part of the organic hydroperoxide containing reaction product with a basic aqueous solution and separating hydrocarbonaceous phase containing organic hydroperoxide from basic aqueous phase,
(c) washing with water at least part of the separated hydrocarbonaceous phase containing organic hydroperoxide and separating hydrocarbonaceous phase containing organic hydroperoxide from aqueous phase; and,
(d) optionally repeating step (c) one or more times, in which process solid particles are removed from the reaction product containing organic hydroperoxide and/or basic aqueous solution before use in step (b).

2. The process of claim 1, in which process solid particles are removed by filtering at least part of the reaction product containing organic hydroperoxide and/or at least part of basic aqueous solution before use in step (b) with a filter.

3. The process of claim 2, wherein at least part of the basic aqueous solution which has been separated off in step (b) is filtered and subsequently recycled to step (b).

4. The process of claim 1, wherein the basic aqueous solution contains fresh basic aqueous solution, recycled basic aqueous solution and optionally additional water.

5. The process of claim 1, wherein in step (c) and/or (d) at least part of the separated hydrocarbonaceous phase containing organic hydroperoxide is washed with waste water optionally in combination with clean water.

6. A process for preparing oxirane compounds, which process comprises:
(a) oxidizing an organic compound to obtain a reaction product containing organic hydroperoxide;
(b) treating at least part of the organic hydroperoxide containing reaction product with a basic aqueous solution and separating hydrocarbonaceous phase containing organic hydroperoxide from basic aqueous phase,
(c) washing with water at least part of the separated hydrocarbonaceous phase containing organic hydroperoxide and separating hydrocarbonaceous phase containing organic hydroperoxide from aqueous phase;
(d) optionally repeating step (c) one or more times;
(e) contacting at least part of the hydrocarbonaceous phase containing alkylaryl hydroperoxide obtained in step (c) and/or (d) with olefin and catalyst to obtain alkylaryl hydroxide and oxirane compounds; and,
(f) separating at least part of the oxirane compound from the alkylaryl hydroxide,
in which process solid particles are removed from the reaction product containing organic hydroperoxide and/or basic aqueous solution before use in step (b).

7. The process of claim 6, which process further comprises:
(g) dehydrating at least part of the alkylaryl hydroxide obtained in step (f) to obtain an alkenyl aryl compound and water.

8. The process of claim 6, which process further comprises:
(g) converting at least part of the alkylaryl hydroxide obtained in step (f) via hydrogenolysis to obtain an alkylaryl compound.

9. The process of claim 6, wherein solid particles are removed by filtering at least part of the reaction product containing organic hydroperoxide and/or at least part of basic aqueous solution before use in step (b) with a filter.

10. The process of claim 9, wherein the filter has openings of 50 micrometers or less.

11. The process of claim 9, wherein at least part of the basic aqueous solution which has been separated off in step (b) is filtered and subsequently recycled to step (b).

12. The process of claim 6, wherein the basic aqueous solution contains fresh basic aqueous solution, recycled basic aqueous solution and optionally additional water.

13. The process of claim 6, wherein in step (c) and/or (d) at least part of the separated hydrocarbonaceous phase containing organic hydroperoxide is washed with waste water optionally in combination with clean water.
